(19) United States
(12) Patent Application Publication
(10) Pub. No.: US 2007/0037236 A1
(43) Pub. Date: Feb. 15, 2007

(54) Factor Xa-based heparin assay using a heparin-modifying component

(75) Inventors: Wolfgang Klein, Marburg (DE); Norbert Zander, Marburg (DE)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: DADE BEHRING MARBURG GmbH

(21) Appl. No.: 11/502,357

(22) Filed: Aug. 11, 2006

(30) Foreign Application Priority Data
Aug. 12, 2005 (DE) 10 2005 038 418.8

(51) Int. Cl. C12Q 1/56; C12Q 1/34

(52) U.S. Cl. 435/13; 435/18

(57) ABSTRACT

The present invention is concerned with the diagnosis of coagulation and relates to a method for determining the heparin activity in a sample, where the sample is initially incubated with a heparin-modifying component, and then the heparin-dependent FXa inactivation is measured.

### CROSS REFERENCE TO RELATED APPLICATIONS

This application claims the benefit under 35 U.S.C. 119 of the Patent Application 10 2005 038 418.8, filed in Germany on Aug. 12, 2005. The disclosure of this application is incorporated by reference in its entirety.

### FIELD OF THE INVENTION

The present invention is concerned with the diagnosis of coagulation disorders and relates to an in vitro method for determining the heparin activity in a sample.

### BACKGROUND OF THE INVENTION

Heparins are negatively charged, polysulfated glucosaminoglycan chains of varying length and are among the most widespread anticoagulant substances which inhibit coagulation immediately and concentration-dependently in vivo and in vitro. Depending on the chain length, a distinction is essentially made between so-called unfractionated, high molecular weight heparins (UFH, UF heparins) having a molecular weight of between 3000 and 30 000 daltons and so-called fractionated, low molecular weight heparins (LMWH, LMW heparins) having a molecular weight of between about 4000 and 9000 daltons. Fractionated heparins are generally obtained from unfractionated heparin by enzymatic or chemical cleavage or by chromatography. Depending on the cleavage method, the resulting products differ in physicochemical properties such as average chain length, molecular weight distribution, degree of sulfation or glycan modifications, which influence the biological activity of these products. Heparin derivatives and heparinoids are enzymatically and/or chemically modified glucosaminoglycan chains whose anticoagulant properties can be modified biotechnologically through targeted influencing of glycan chain length, sulfation pattern and acetylation pattern or through mixing different glucosaminoglycans, as, for example, in the case of danaparoid sodium, a mixture of glycosaminoglycans consisting predominantly of heparan sulfate and a smaller proportion of dermatan sulfate and chondroitin sulfate.

The anticoagulant effect of all heparins derives from their formation of complexes with antithrombin (AT, antithrombin III), the most important plasmatic inhibitor of activated coagulation factors. Antithrombin belongs to the group of serine protease inhibitors (serpins) and inhibits the coagulation factors thrombin (factor IIa, FIIa) and factor Xa (FXa) and, to a small extent, also the other serine proteases FIXa, FXIa, FXIIa, kallikrein and plasmin. The binding of heparin to antithrombin results in a change in the antithrombin conformation, which enhances many-fold the inhibitory effect of antithrombin. The binding site in heparin molecules which is responsible for the binding to antithrombin consists of a characteristic pentasaccharide sequence. A completely synthetic form of this pentasaccharide (fondaparinux) is employed just like UFH or LMWH for the medical inhibition of coagulability.

Unfractionated heparins and fractionated heparins, heparin derivatives, heparinoids and pentasaccharides differ in their anticoagulant effect. Whereas UFH inhibit thrombin and FXa equally, LMWH exhibit predominantly an FXa-inhibiting effect and only to a minor extent a thrombin-inhibiting effect. Pentasaccharides such as fondaparinux selectively inhibit FXa and show no thrombin inhibition at all.

Depending on the indication, heparins are employed prophylactically or therapeutically, e.g. for prophylaxis of thromboembolism in situations of risk, such as surgery or during immobilization, for the therapy of manifested thromboembolic events or in extracorporeal therapeutic methods such as haemodialysis, cardiopulmonary bypass or cardiac catheterization investigations. Since the anticoagulant effect of heparin is subject to a number of individual influencing variables such as, for example, body weight or hepatic and renal function, and because an incorrect dosage may induce bleeding complications or thromboembolic complications, monitoring of therapy is indicated for UFH in all patients and for LMWH in patients at risk (renal failure, obesity, pregnancy).

Various methods are known in the art for monitoring heparin therapy and for determining the heparin level.

Therapy and prophylaxis with heparin can firstly be monitored indirectly by checking the coagulability of blood with the aid of conventional global coagulation tests such as, for example, the PT, the APTT and the thrombin time. Comparison of a measured clotting time for a heparinized patient's sample with corresponding reference values from standard plasmas of defined heparin concentration also makes it possible to quantify the heparin level in a patient's sample. Examples of clotting time-based methods for determining heparin are described for example in the patents U.S. Pat. No. 4,067,777 and EP 217 768 B1.

A particular type of a clotting time-based heparin determination is described in EP 259 463 B1. In this method, a clotting time is determined for one aliquot of a patient's sample, while a second aliquot of the sample first is treated with heparinase for complete degradation of the heparin present in the sample, and then clotting time is determined. The difference in the two clotting times is a measure of the amount of heparin present in the sample, which can be read off an appropriate standard curve. However, in literature, global coagulation tests are regarded as unsuitable for determining LMWH, since their reduced antithrombin effect means that the effect on the measured result (clotting time, INR) is not linear and concentration-dependent [see, for example, Harenberg, J. (2004) Is laboratory monitoring of low-molecular-weight heparin therapy necessary? Yes. J. Thromb. Haemost. 2; pages 547-550].

Secondly, heparin therapy or prophylaxis can be monitored with the aid of so-called amidolytic or chromogenic heparin assays which permit precise and sensitive determination of the heparin activity. These methods are based substantially on incubating a heparinized sample with exogenous FXa or thrombin (FIIa) and with an appropriate factor-specific chromogenic substrate, and measuring the heparin-dependent inactivation of the enzymatic FXa or thrombin activity from the conversion of substrate. The amount of liberated chromogenic cleavage product is in inverse proportion to the heparin activity. With the aid of a calibration curve, constructed from measurement of plasmas with known heparin contents, correct quantification of the amount of heparin in a patient's sample is possible. Additional antithrombin addition to the reaction mixture, either in the form of normal plasma or else in purified form, has the effect firstly, by creating an excess, of offsetting different antithrombin concentrations in different patients' samples, and secondly of increasing the sensitivity of the test, especially in low heparin concentration ranges. Diverse variants of such amidolytic heparin assays are commercially available. Examples are described in the patents EP 004 271 A2 and EP 034 320 B1. According to the prior art, chromogenic anti-FXa tests of this type are to be employed preferably in the determination of LMWH [see, for example, Harenberg, J. (2004) in this connection too].

Heparin is normally quantified in international units per milliliter (IU/ml). Since heparins, as extracted biological material, depend greatly on the organism, tissue, extraction conditions inter alia, and the measurable effects on coagulation can be detected only indirectly, a physicochemical definition of effect (unit) has not proved to be practicable. For this reason, appropriate metrological standards have been defined and are used for the measurement and declaration of products as well as new standards. The most common one is the international unit (IU) system supported by the WHO and administered by the National Institute for Biological Standards and Controls (NIBSC, UK) [for a review, see, for example, Mulloy, B. et al. (2000) Characterization of unfractionated heparin: Comparison of materials from the last 50 years. Thromb. Haemost. 84, 1052-1056 and most recent publications on biological standards on the homepage of the NIBSC, www.nibsc.ac.uk].

As already described above, there are differences in the strength of inhibition of the enzymatic activity of factor Xa by the different heparins. For this reason, to calibrate a factor Xa-based assay, it is necessary to construct a separate calibration curve for each heparin to be determined, by using different concentrations of the heparin which is to be quantified in the patient's sample. In order to be able to carry out the precise determination of heparin in a sample at all, it is thus necessary to know which heparin product is used to treat the patient, so that the measured values can be quantified with the correct calibration curve. The disadvantages of this method are that the construction of different calibration curves causes costs for labor and materials, that there is always a risk of incorrect information about the heparin product used on the patient, resulting in the use of an incorrect calibration curve for the quantification and consequently in an incorrect interpretation of the test results, and that reliable heparin determination is not possible if no information is given about the product used to treat the patient.

### SUMMARY OF THE INVENTION

The present invention was thus based on the object of providing a method for heparin determination which makes it possible to dispense with the generation of heparin product-specific calibration curves and to use a universal calibration curve for all heparins. The achievement of the object according to the invention consists of providing the articles and methods described in the claims.

The present invention relates to an in vitro method for determining heparins, i.e. UF heparin, LMW heparin, heparin derivatives, heparinoids and pentasaccharides in liquid samples, in particular in body fluid samples such as blood, plasma, serum or urine, where the heparin activity is determined from the heparin-dependent factor Xa inactivation. Surprisingly, it has now been found that incubation of a heparinized sample which comprises unfractionated or fractionated heparin, a heparin derivative, a heparinoid or a synthetic pentasaccharide with a heparin-modifying component leads to offsetting of the differences in the FXa-inhibiting effect of the different heparins, but the remaining FXa-inhibiting effect still correlates with the heparin content originally present in the sample, and thus quantification with a single calibration curve is possible (see also FIG. 3).

### BRIEF DESCRIPTION OF THE FIGURES

FIG. 1A shows the anti-coagulation activity of different forms of heparin in the presence of the heparin-modifying component heparinase I. The anti-coagulation activity is reflected in the inhibition of factor Xa protease activity by antithrombin. The curves show the change in the measured signals (ΔOD/min) over the incubation time. Liquemin® is an unfractionated, high molecular weight heparin and Fragmin® is a fractionated, low molecular weight heparin. SHP is standard human plasma.

FIG. 1B shows a similar experiment as in FIG. 1A, but with a higher amount of heparinase I, as indicated.

FIG. 2A shows the effects of increasing concentrations of the heparin-modifying component heparinase I on the anti-coagulation activity of the heparin Liquemin®. The curves show the change in the measured signals (ΔOD/min) with various heparin concentrations in the citrated plasma.

FIG. 2B shows a similar experiment as in FIG. 2A, but with the heparin Fragmin®.

FIG. 3 shows the anti-coagulation activities of different heparins (Liquemin® and Fragmin®) in the absence or presence of the heparin-modifying component heparinase I. The curves show the change in the measured signals (ΔOD/min) for different heparin concentrations in citrated plasma.

### DETAILED DESCRIPTION OF THE INVENTION

A heparin-modifying component means in the context of the present invention a substance or a mixture of substances which is able to remove oligosaccharide units from heparin and which, when used in an FXa-based heparin assay, exerts no disadvantageous effects on constituents of the sample material or of the other added reagents, so that quantitative determination of the heparin-dependent FXa inhibition is still possible in the presence of the substance. The suitability of a substance for use as heparin-modifying component in the present method of the invention can be ascertained for example by carrying out a FXa-based heparin assay in the presence and, in parallel, in the absence of the substance to be investigated. Suitable substances can be recognized by the fact that the absolute anti-FXa activity measured in UFH-containing samples in the presence of the substance is lower than in the absence of the substance, although the anti-FXa activity remaining in the presence of the substance still correlates with the heparin activity originally present in the sample, thus making correct quantification of the heparin activity possible.

Heparin-modifying components suitable for the method of the invention are in particular enzymes because they act specifically and under moderate, virtually physiological conditions, thus making it possible to minimize or preclude disadvantageous secondary effects on the test method per se. Enzymes from the groups of glucuronidases and heparinases, which are capable of truncation and modification of glycosaminoglycan chains, are particularly suitable. Glucuronidases are heparin-hydrolyzing enzymes of diverse origin, e.g. from Escherichia coli, bovine liver or mollusks, which truncate glucosaminoglycan chains without chemical modification. Heparinases, also called heparin lyases, are heparin-specific enzymes which are synthesized naturally by microorganisms such as, for example, Flavobacterium heparinum, and have an eliminative reaction mechanism, i.e. also chemically modify terminal sugar groups in the cleavage reaction. Particularly suitable for use as heparin-modifying component in the method of the invention are heparinase I, heparinase II and heparinase III, which can be either directly obtained from, for example, F. heparinum cultures or else expressed recombinantly, for example in Escherichia coli. Likewise suitable as heparin-modifying component are mixtures of various heparinases and/or glucuronidases. Methods for obtaining endogenous heparinases from F. heparinum and for production of the enzymes by genetic manipulation are described in the prior art, e.g. in U.S. Pat. No. 5,714,376 and in EP 670 892 B1 and the publications cited therein.

Whereas heparin-modifying or -degrading enzymes are used in the art to neutralize heparin by complete degradation, the heparin-modifying components are employed in the present method in such a way that they do not completely degrade the heparin present in a sample, but that the heparin present in the sample is merely modified so that quantification of the antithrombin-activating properties is still possible. For this purpose, the heparin-modifying component is incubated with the sample preferably in a concentration of from 0.05 to 5.0 U/ml, particularly preferably in a concentration of from 0.25 to 1.25 U/ml in the mixture, before FXa is added. In this connection, 1 unit corresponds to the amount of enzyme required to eliminate 1 μmol of unsaturated oligosaccharides from heparin (from porcine mucosa; about 109 IU/mg) per minute at 30° C. and pH 7.0.

In the present method, the sample is incubated with the heparin-modifying component for a defined period before factor Xa is added. The sample is incubated with a heparin-modifying component before the addition of FXa preferably for a period of from 10 to 900 seconds, particularly preferably for a period of from 15 to 90 seconds, very particularly preferably for a period of from 20 to 60 seconds. Shorter and longer incubation times are possible, it being necessary to match the incubation time primarily with the employed concentration of heparin-modifying component and the chosen reaction temperature. Normally a reaction temperature of 37° C. is preferred for determining physiologically relevant parameters in the diagnosis of coagulation. Lower or higher reaction temperatures can likewise be used as long as the skilled worker includes in the calculation the thermodynamic effects of which he is aware, e.g. on the kinetics of biochemical reactions.

In a preferred embodiment of the method of the invention, a citrated plasma sample is initially mixed with a reagent which comprises antithrombin. This may take the form for example of normal plasma or else of purified antithrombin. Antithrombin is preferably added in excess, so that more antithrombin is present in the mixture than necessary to inhibit FXa. In the event that the sample is pretreated with an antithrombin reagent, the heparin-modifying component can preferably be present in this reagent.

Incubation of the sample with the heparin-modifying component and, where appropriate, with antithrombin is followed by addition of factor Xa to the reaction mixture. This factor Xa may be a human, bovine or a recombinant variant thereof. In a particularly preferred embodiment, the FXa reagent can additionally comprise dextran sulfate. It is likewise possible to add the dextran sulfate independently of the FXa reagent to the sample.

After an incubation period during which the added factor Xa is inactivated as a function of the heparin present in the sample or of the heparin derivative modified according to this invention, a substrate is added to the reaction mixture. Chromogenic substrates which release a dye on exposure to factor Xa are preferably used. This dye may be one which can be determined in the visible region of the spectrum, a fluorescent dye or a dye which can be determined in the UV region. Peptides which have a dye residue linked by an amide linkage to the carboxy group of an arginine residue are preferably used. Particularly suitable for this purpose are p-nitroanilide groups (pNA) and 5-amino-2-nitrobenzoic acid derivatives (ANBA), and dyes which are derived therefrom by substitution and can be quantified by a photometric measurement at a wavelength of 405 nm after the peptide position has been released. Examples of suitable substrates are Bz-lle-Glu-Gly-Arg-pNA and Z-D-Leu-Gly-Arg-ANBA-methylamide.

The method can be evaluated as kinetic or endpoint determination. In the kinetic method, the reaction, i.e. the remaining factor Xa activity, is quantified as a measure of the heparin activity present, on the basis of the conversion rate of chromogenic substrate. In the endpoint determination, the reaction is stopped after a predetermined measurement time by adding an acid, e.g. acetic acid, and the amount of released dye is measured.

In a preferred embodiment of the method of the invention, the heparin activity of a sample is quantified irrespective of the nature of the heparin present in the sample by means of a universal calibration curve which is suitable for all heparin variants. The evaluation takes place on the basis of a reference curve which has been generated with the aid of a calibrator solution which comprises a defined amount of heparin, and/or with the aid of dilutions of this calibrator solution. In contrast to methods known in the art, it is unnecessary in the method of the invention for the heparin preparation used for the calibration to be the same as that which is to be determined in a patient's sample. The method of the invention allows any heparin, unfractionated or fractionated heparin or a pentasaccharide to be used for the calibration and the heparin activity of any patient's sample to be quantified using the generated calibration curve, irrespective of the preparation administered. For example, Liquemin® (Roche, Basle, Switzerland), an unfractionated, high molecular weight heparin from porcine mucosa, or Fragmin® (Pharmacia, Kalamazoo, USA), a fractionated, low molecular weight heparin, can be used to generate a universal calibration curve suitable for all heparin variants. It is preferred for a UF heparin or an LMW heparin to be diluted to 10 IU/ml with isotonic saline solution and finally diluted with a heparin-free pooled or standard plasma so that, by means of the method of the invention, a reference curve can be generated for the entire therapeutic range.

The method of the invention is suitable both for being carried out manually and for use in automatic analyzers.

The method of the invention is suitable only conditionally for determining the heparin activity in a sample which additionally comprises a substance which inhibits the heparin-modifying component used for carrying out the method. The patent document WO 01/46385 A1 describes for example the compounds CRM646-A and CRM646-B which act as heparinase inhibitors and which appear suitable for therapeutic use. The heparin-modifying effect may be impaired on determination according to the invention of the heparin activity in a sample which comprises a substance which inhibits the heparin-modifying component used. Quantification of the heparin content of such a sample using a calibration curve previously constructed with the aid of the method of the invention would very probably lead to erroneous determinations. The method of the invention is accordingly preferably suitable for determining the heparin activity in a liquid sample which is free of substances which inhibit the heparin-modifying component used in the method. In preferred embodiments, the sample is free of glucuronidase inhibitors and/or free of heparinase inhibitors, such as, for example, CRM646-A and CRM646-B (WO 01/46385 A1). Samples which are free of heparinase and/or glucuronidase inhibitors within the meaning of the present invention are, for example, body fluid samples which are derived from one or more humans or animals which have neither been treated therapeutically in vivo with such an inhibitor nor mixed in vitro with such an inhibitor.

A further aspect of the present invention relates to a test kit for carrying out the FXa-based heparin assay of the invention, where the test kit comprises at least two reagents, one of which comprises a heparin-modifying component, and the other FXa. The reagents may additionally comprise preservatives and be provided either as liquid reagents or as lyophilizates.

The reagent which comprises the heparin-modifying component may additionally comprise a further component which is required to carry out an FXa-based heparin assay, such as, for example, antithrombin or a substrate. Depending on the test design, the sequence of addition of the individual components to the sample may differ. In some cases, the detection reaction will be started by adding the substrate, and in other cases by adding FXa. Attention must be paid to the heparin-modifying component being present in a reagent which is mixed with the sample before addition of FXa. A particularly preferred embodiment of a test kit of the invention comprises a reagent that comprises the heparin-modifying component together with antithrombin.

Besides the reagent which comprises the heparin-modifying component, and the reagent which comprises FXa, a test kit of the invention may additionally comprise further reagents such as, for example, a reagent which comprises a factor Xa substrate, and/or a reagent which comprises antithrombin. A preferred FXa reagent may additionally comprise dextran sulfate. Another preferred reagent may comprise antithrombin together with dextran sulfate.

The reagents in the test kit of the invention can be provided in liquid or lyophilized form. In the event that some or all of the reagents in the test kit are in the form of lyophilizates, the test kit may additionally comprise the solvents necessary to dissolve the lyophilizates, such as, for example, distilled water, suitable buffers and/or standard human plasma.

The following examples serve to illustrate the method of the invention and are not to be regarded as restrictive.

### EXAMPLES

### Example 1

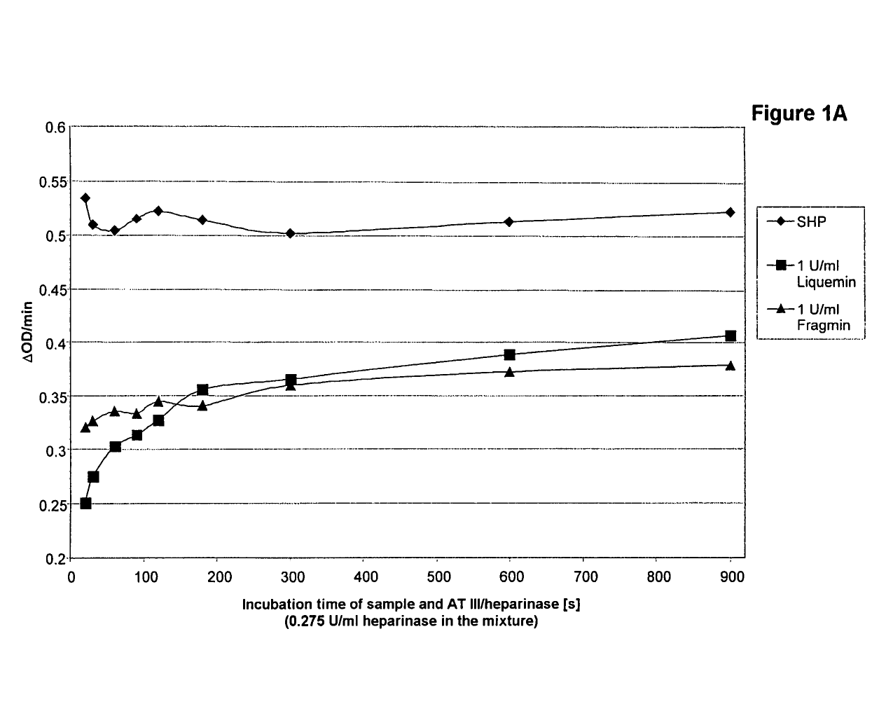

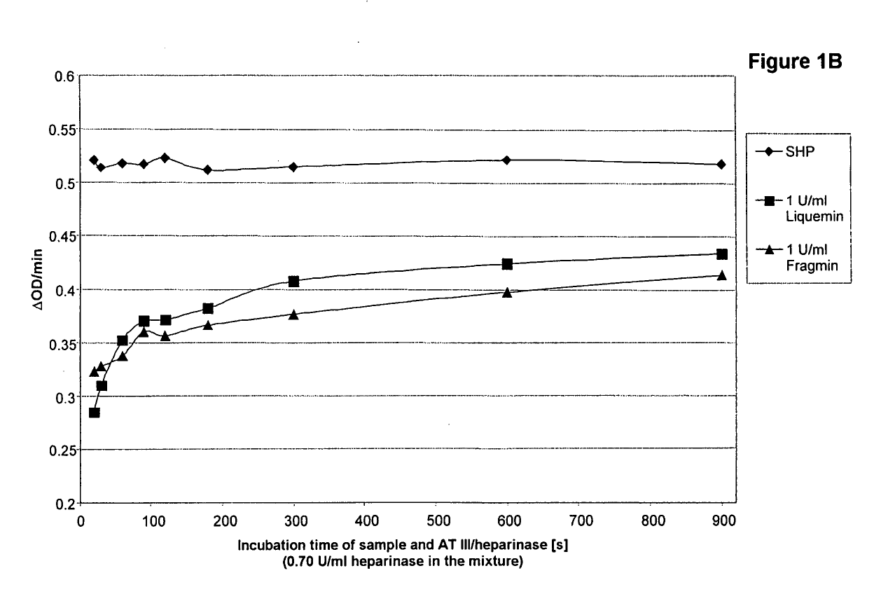

Lyophilized and reconstituted human citrated plasma was spiked with 1 IU/mi Liquemin® (Roche, Basle, Switzerland), a UF heparin, or with Fragmin® (Pharmacia, Kalamazoo, USA), an LMW heparin. 20 μl samples of plasma were incubated with 20 μl of antithrombin reagent (AT reagent; lyophilized and reconstituted, 1 IU/ml) which additionally contained 0.55 U/ml (FIG. 1A) or 1.4 U/ml (FIG. 1B) heparinase I (Dade® Hepzyme®, Dade Behring Marburg GmbH, Marburg, Germany) at 37° C. for 20 to 900 seconds. The reaction mixture was then mixed with 170 μl of factor Xa reagent [lyophilized and reconstituted human FXa with addition of TRIS (6 g/l), EDTA (0.74 g/l) and sodium chloride (12 g/l)] and incubated at 37° C. for 1 minute. The reaction was started by adding 40 μl of substrate reagent (Z-D-Leu-Gly-Arg-ANBA-methylamide, 4 mM), and the formation of ANBA was detected at a wavelength of 405 nm and 37° C. in an automatic measuring instrument, Sysmex® CA-1500 (Dade Behring Marburg GmbH, Marburg, Germany). The change in extinction per minute (ΔOD/min) has been depicted in FIG. 1 for the various times of incubation of plasma with AT-heparinase reagent.

FIG. 1 shows the change in the measured signals (ΔOD/min) over the incubation time with two different heparinase activities in the mixture for plasmas with UFH (Liquemin®) and LMWH (Fragmin®). A heparin-free standard human plasma (SHP) was measured in each case as control. This plasma without heparin showed no change in extinction as a function of the time of incubation of the sample with AT-heparinase reagent. With the heparin-containing plasmas there is an increase in the change in extinction with increasing incubation time. After about 300 seconds, the change in extinction reaches a plateau which is distinctly lower than that of the heparin-free plasma. Even extending the incubation of the sample with heparinase to 900 seconds cannot eliminate the anti-FXa activity of the investigated heparins completely. The heparinase contents employed here modulate the absolute height of the plateau reached to only a small extent, compare FIG. 1A (0.275 U/ml heparinase in the mixture before addition of FXa) and FIG. 1B (0.7 U/ml heparinase in the mixture before addition of FXa).

### Example 2

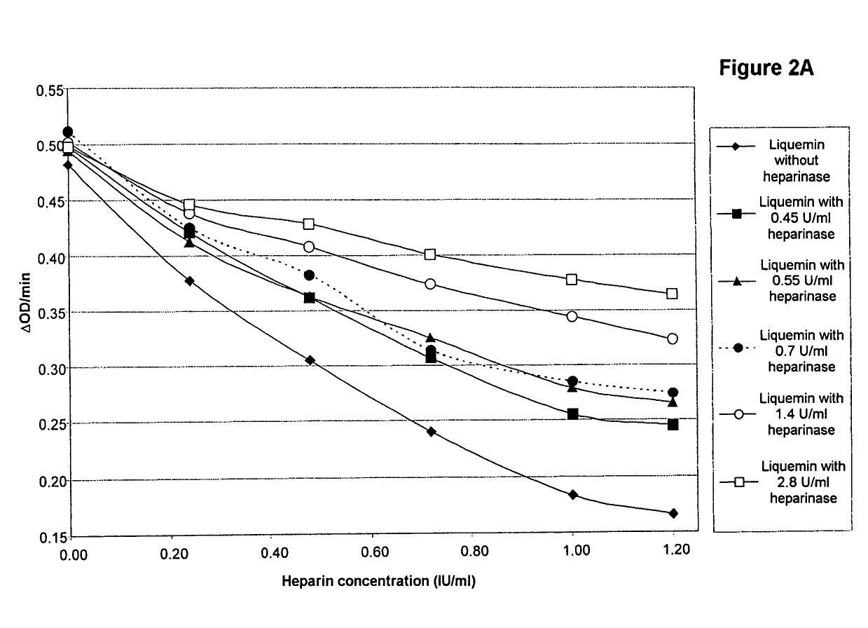

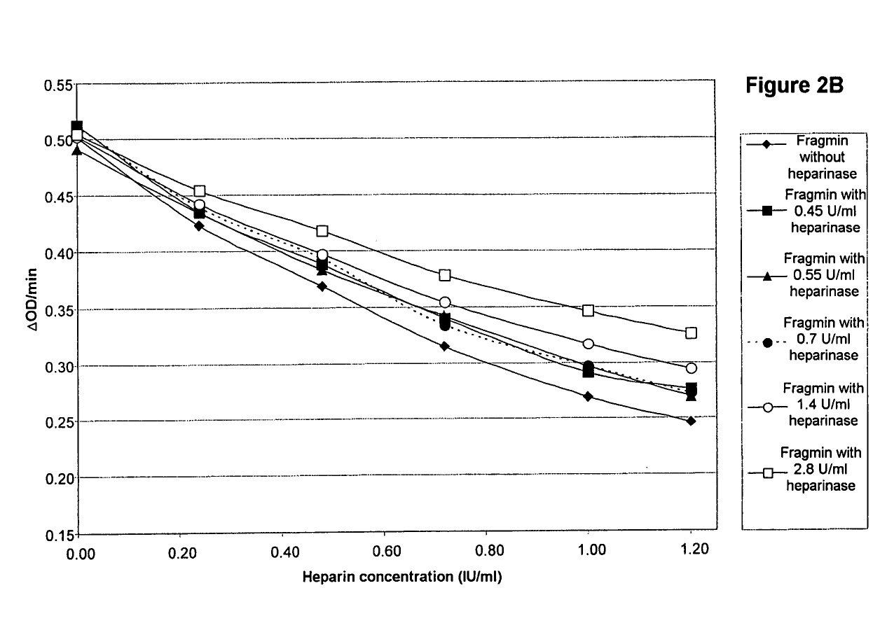

Lyophilized and reconstituted human citrated plasma was spiked with 0.24, 0.48, 0.72, 1 and 1.2 IU/ml of each of Liquemin® (FIG. 2A) or Fragmin® (FIG. 2B). AT reagent (lyophilized and reconstituted, 1 IU/ml) was mixed with increasing concentrations of heparinase I (Dade® Hepzyme®) from 0 to 5.5 U/ml. 20 μl samples of plasma were each mixed with 20 μl of AT-heparinase reagent and incubated at 37° C. for 20 seconds. The reaction mixtures were then mixed with 170 μl of factor Xa reagent (see Example 1) and incubated at 37° C. for 1 minute. The reactions were started by adding 40 μl of substrate reagent (see Example 1), and the formation of ANBA was detected automatically as already described in Example 1. The change in extinction per minute (ΔOD/min) for the various reaction mixtures of plasma with AT-heparinase reagent is depicted in FIG. 2. Calibration curves with different heparinase activities were simulated in this way.

FIG. 2 depicts the change in the measured signals (ΔOD/min) with various heparin concentrations in the citrated plasma. The different curves reflect the different heparinase activities in the mixture before adding FXa. The plasmas were spiked with UFH (Liquemin®) in FIG. 2A and with LMWH (Fragmin®) in FIG. 2B. The figures show that the calibration curves become less steep both for UFHs and for LMWHs with increasing heparinase contents but, on the other hand, with identical heparin contents or activities of Liquemin® or Fragmin®, the initial rate of degradation is greater for UF heparin (Liquemin®) than for LMW heparin (Fragmin®). Even with the highest heparinase concentration depicted in the example, the calibration lines show a linearly monotonic course depending on the initial concentration of the heparins. The anti-FXa activity of the heparins is not completely eliminated by the highest heparinase activity chosen in the example even with a heparinase incubation time of 20 seconds at 37° C.

### Example 3

An exemplary test design was chosen here in order to illustrate the potential of a uniform modification of UFH and LMWH and the possibility of a common calibration curve.

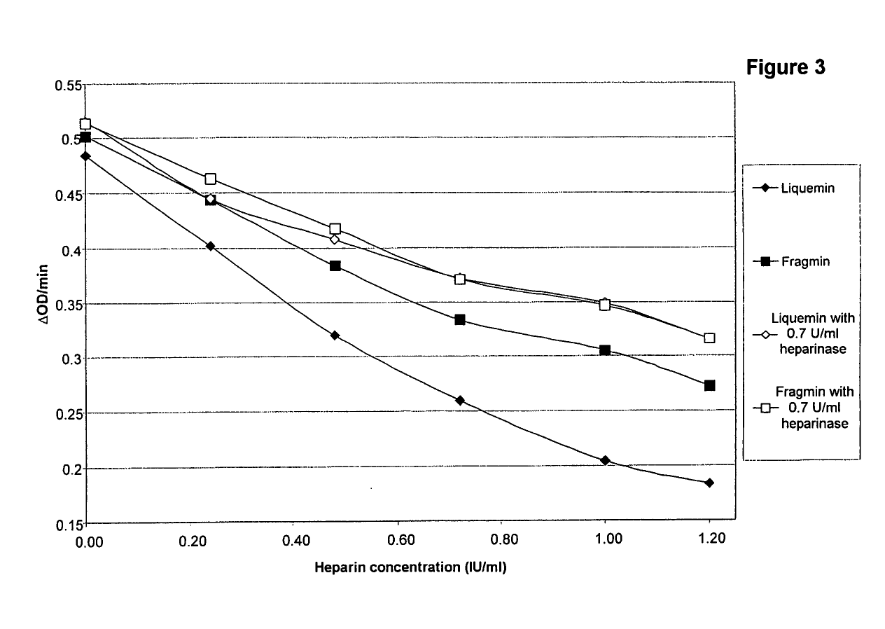

Lyophilized and reconstituted human citrated plasma was spiked with 0.24, 0.48, 0.72, 1.00 and 1.20 IU/ml of each of Liquemin® or Fragmin®. AT reagent (lyophilized and reconstituted, 1 IU/ml) was mixed with heparinase I (Dade® Hepzyme®) (1.4 U/ml), while AT reagent without added heparinase served as control. 20 μl samples of plasma were each mixed with 20 μl of AT reagent and incubated at 37° C. for 20 seconds. The reaction mixtures were then mixed with 170 μl of factor Xa reagent (see Example 1) and incubated at 37° C. for 1 minute. The reactions were started by adding 40 μl of substrate reagent (see Example 1), and the formation of ANBA was detected automatically as already described in Example 1. The change in extinction per minute (ΔOD/min) has been depicted for the various reaction mixtures of plasma with AT-heparinase reagent in FIG. 3.

FIG. 3 depicts the change in the measured signals (ΔOD/min) for different heparin concentrations in citrated plasma. The curves in each case describe a combination of heparin (UFH or LMWH) and a chosen heparinase activity (0 U/ml as basic test corresponding to the prior art, and 0.7 U/ml corresponding to the invention described herein). FIG. 3 shows the divergence of UFH and LMWH calibration curves without added heparinase for the examples of Liquemin® and Fragmin® (black symbols). Carrying out the calibrations with heparinase-containing AT reagent leads to the calibration curves for the various heparins coming close together (white symbols). Although the gradients of the calibration curves become smaller in the example shown through the use of the heparin-modifying components, there is still a clear linear association between the measured signals and the heparin concentration in the plasma, which permits correct determination of heparin concentrations in plasma samples on the basis of these calibration curves.

The skilled worker is able through his general expert knowledge to change the gradient of the calibration curve by routine modifications in the test procedure (e.g. changing the sample volume, changing the FXa-inactivation time, changing the reaction temperature, the pH, the ionic strength or other parameters) and adapt it to the test requirements.

1. A method for determining the heparin activity in a liquid sample, wherein the heparin-dependent inactivation of added factor Xa is quantified, comprising:
(a) providing a liquid sample;
(b) adding a heparin-modifying component to the sample; and
(c) adding factor Xa to the sample after addition of the heparin-modifying component.

2. The method of claim in claim 1, wherein the heparin-modifying component comprises:
(a) a heparinase;
(b) a glucuronidase;
(c) a mixture of different heparinases;
(d) a mixture of different glucuronidases; or
(e) a mixture of at least one heparinase and at least one glucuronidase.

3. The method of claim 2, wherein the heparin-modifying component comprises:
(a) heparinase I;
(b) heparinase II;
(c) heparinase III; or
(d) a mixture of any of (a), (b), and (c).

4. The method of claim 1, wherein the heparin-modifying component is used in a concentration range from 0.05 to 5.0 U/ml.

5. The method of claim 1, wherein the sample is incubated with a heparin-modifying component for 10 to 900 seconds before addition of factor Xa.

6. The method of claim 1, wherein the heparin-dependent inactivation of added factor Xa is quantified in the presence of added antithrombin.

7. The method of claim 6, wherein the antithrombin is added to the sample at the same time as the heparin-modifying component.

8. The method of claim 1, wherein additionally dextran sulfate is added to the sample.

9. The method of claim 8, wherein the dextran sulfate is added at the same time as factor Xa.

10. The method of claim 1, wherein the sample is a body fluid sample.

11. The method of claim 1, wherein further the heparin activity in the sample is quantified irrespective of the nature of the heparin present in the sample by means of a universal calibration curve which is suitable for all heparin variants and which has been constructed using any heparin.

12. A test kit which comprises:
(a) a reagent comprising a heparin-modifying component; and
(b) a reagent comprising factor Xa.

13. The test kit of claim 12, which further comprises a reagent comprising a factor Xa substrate.

14. The test kit of claim 12, which further comprises a reagent comprising antithrombin.

15. The test kit of claim 12, wherein the reagent (a) further comprises antithrombin.

16. The test kit of claim 15, wherein the reagent (b) further comprises dextran sulfate.

17. The test kit of claim 14, wherein the antithrombin-containing reagent further comprises dextran sulfate.

18. The test kit of claim 12, wherein one or more of the reagents are lyophilized.

19. The method of claim 1, wherein the method is carried out using a test kit.

20. The method of claim 1, wherein the heparin-modifying component is used in a concentration range from 0.25 to 1.25 U/ml.

21. The method of claim 1, wherein the sample is incubated with a heparin-modifying component for 15 to 90 seconds before addition of factor Xa.

22. The method of claim 1, wherein the sample is incubated with a heparin-modifying component for 20 to 60 seconds before addition of factor Xa.

23. The method of claim 19, wherein the test kit comprises:
(a) a reagent comprising a heparin-modifying component; and
(b) a reagent comprising factor Xa.

24. The method of claim 10, wherein the body fluid is blood, blood plasma, serum or urine.
